(19) United States
(12) Patent Application Publication
(10) Pub. No.: US 2022/0111579 A1
(43) Pub. Date: Apr. 14, 2022

(54) HYBRID PRINTING PLATFORM FOR 3D BIOPRINTING OF LIVE ORGANS

(71) Applicant: Applied Materials, Inc., Santa Clara, CA (US)

(72) Inventors: Daihua ZHANG, Los Altos, CA (US); Uma SRIDHAR, Sunnyvale, CA (US); Hou T. NG, Campbell, CA (US); Sivapackia GANAPATHIAPPAN, Los Altos, CA (US); Nag B. PATIBANDLA, Pleasanton, CA (US)

(21) Appl. No.: 17/070,854

(22) Filed: Oct. 14, 2020

(51) Int. Cl. B29C 64/112; B29C 64/264; B29C 64/209; B29C 64/245; B29C 64/232; B33Y 70/00; A61F 2/02

(52) U.S. Cl. CPC B29C 64/112; B29C 64/264; B29C 64/209; B33Y 10/00; B29C 64/232; B33Y 70/00; A61F 2/02; B29C 64/245

(57) ABSTRACT

A method of forming a three dimensional object includes dispensing droplets of an electromagnetic energy curable liquid onto a surface to form a plurality of layers of the three dimensional object in liquid form, wherein each droplet forms a layer of liquid on the surface which is larger than a minimum feature size of a structure to be formed by curing the curable liquid, and directing electromagnetic energy capable of curing the liquid and having a beam width intersecting the layer of liquid which is at least as small as the smallest feature of a structure to be formed in the curable liquid.

### BACKGROUND

### Field

A 3D bio-printing scheme that simultaneously allows for micrometer (micron) scale structural resolution and material heterogeneity in a 3D printed object. More particularly, the present disclosure relates to a unique hybrid 3D printing scheme which combines the high-resolution of digital curing technology with the multi-material deposition capability of an inkjet type printer into one platform.

### Description of the Related Art

Objects desired be manufactured using 3D printing techniques, for example human organs, consist of tissue architectures with different material compositions and physical dimensions having material feature sizes as small as micrometer or micron sizes. There are 3D printing technologies such as stereolithography, also known as “SLA” and digital lithographic printing, also known as “DLP” that can provide high resolution, i.e., small features sizes, of printed structures from a light sensitive material layer of a single composition. Inkjet and fused deposition manufacturing, also knowns as “FDM” technologies allow multi-material processing capabilities, but cannot meet the small feature resolution requirements of certain resulting structure created by 3D printing using these technologies. However, none of these existing solutions can meet both requirements at the same time, i, e., both high resolution printing of small features in the micrometer (micron) scale or size of feature, and individual printed layers of slices having material heterogeneity, or multiple different materials at the same level or slice of the printed material. The present invention proposes a new 3D printing scheme to overcome this challenge. The new 3D printing scheme enables micrometer-scale structural resolution of printed structure, and material heterogeneity, at the same time.

### SUMMARY

In a first aspect, a method of forming an artificial organoid having bioactive cells includes dispensing droplets of an electromagnetic energy curable bioink onto a surface to form a plurality of layers of the organoid in liquid form, wherein the droplets of bioink contain bioactive cell types therein of a structure to be formed in the organoid, and each droplet form a layer of bioink on the surface which is larger than a minimum feature size of a structure to be formed by curing the curable bioink, and directing electromagnetic energy capable of curing the bioink having the bioactive cells into the layer of bioink, the electromagnetic energy having a beam width intersecting the bioink which is at least as small as the smallest feature of a structure to be formed in the organoid.

In an additional aspect, an apparatus useful for forming an artificial organoid having bioactive cells includes a liquid dispensing device capable of dispensing droplets of an electromagnetic energy curable bioink onto a surface to form a slice of the organoid in liquid form, wherein the droplets of bioink contain bioactive cell types therein of a structure to be formed in the organoid, and a droplet forms an area larger than the smallest feature of the structure to be formed in the organoid, and an electromagnetic beam directing device configured to direct electromagnetic energy capable of curing the bioink with the bioactive cells, the electromagnetic energy having a beam area intersecting the bioink which is smaller in width than the smallest feature of a structure to be formed in the organoid.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 is an isometric view of an organ printer capable of dispensing an electromagnetic energy curable material onto carrier mounted on a moveable stage and of curing that material.

FIG. 2 is a schematic view of a substrate carrier tray or base on which a 3D bioprinted organ can be formed.

FIG. 2A is an enlarged view of a corner of a substrate carrier tray or base on which a 3D bioprinted organ can be formed of FIG. 2, within the dashed outline circle of FIG. 2.

FIG. 3 is a schematic view of a branched lumen.

FIG. 4 is a sectional view of the branched lumen of FIG. 3.

FIG. 5 is an isometric view three slices or sublayers of a branched lumen of FIG. 3.

FIG. 6 is a plan view of a portion of a theoretical grid useful for bioink printed on a substrate.

FIG. 7 is a plan view of a portion of FIG. 6.

FIG. 8 is an isometric view of a pint head useful to dispense bioink.

FIG. 9 is a plan view of the underside of the print head of FIG. 8.

FIG. 10 is a plan view of an underside of the output head of the digital lithographic printer of FIG. 1.

FIG. 11 is a plan view of a bioink slice being printed over a previously printed and cured bioink slice.

FIG. 12 is a plan view of the bioink slice being printed over a previously printed and cured bioink slice, showing a feature in the slice being printed being formed as a continuation of a feature in the previously printed and cured slice.

FIG. 13 is a plan view of the slice being printed in FIG. 12, now in a cured state.

FIG. 14 is a plan view of a slice being printed over the printed and cured slice of FIG. 13.

FIG. 15A is a sectional view of a small portion of an artificial organoid being fabricated, and containing a plurality of bioinks deposited on a carrier.

FIG. 15B is a sectional view of the of FIG. 15A small portion of an artificial organoid being fabricated, and containing a plurality of bioinks having at least a portion thereof in a cured state.

FIG. 15C is a sectional view of the organoid of FIG. 15B after being washed to remove uncured bioink therefrom.

FIG. 15D is a sectional view of the organoid of FIG. 15C after being matured or repaired into functional domains.

FIG. 16 is a schematic view of the types of structures a 3D bioprinting device may manufacture.

FIG. 17 is a flowchart showing the acts in the manufacture on the bioprinted organ in FIG. 15D.

FIGS. 18A to 18D are isometric schematic views of an organoid being fabricated in a tray, showing a sacrificial material surrounding, and supporting, the volume of the organoid.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

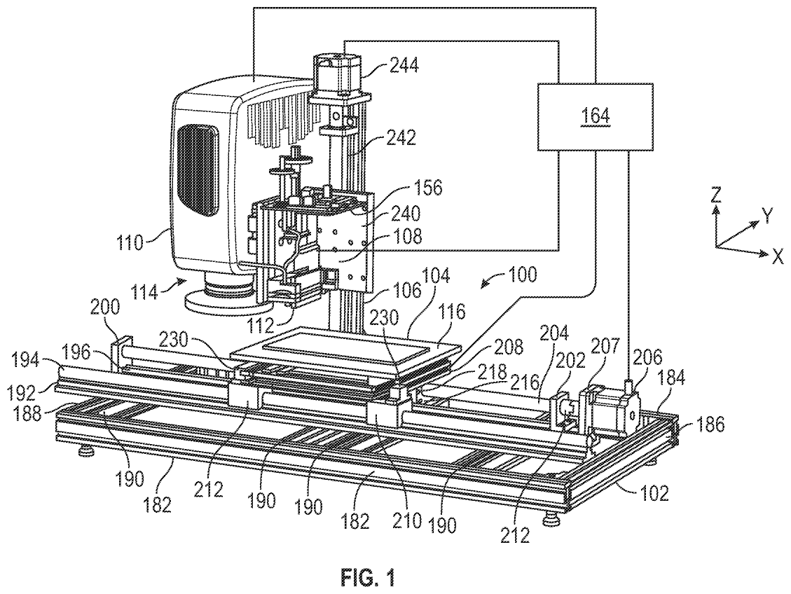

Referring initially to FIG. 1, an inkjet type printer 112 configured to dispense an electromagnetic energy curable material, here a bioink containing living or bioactive cells and a material curable by directing electromagnetic energy therein, onto carrier 124 mounted on a moveable stage 104, and to cure that material, is shown. Here, the dispense and cure station 100 generally includes a base 102, a stage 104 moveable in the X-Y plane of Figure X, an upright support 106, a dispense station 108 and curing station 110 operatively connected to the upright support 106. In this configuration, the dispense station 108 and the cure station 100 are controllably moveably coupled to the upright support 106 to move in the Z direction of FIG. 1, but are generally fixed against movement in the X and Y direction by their connection to the upright support 106. In the construct of Figure X, the dispense station 108 is configured as an inkjet type printer 112 capable of dispensing droplets of material capable of being cured by electromagnetic energy, and the curing station is a what is commonly known as a digital lithographic printer 114, configured to output one or more wavelength of electromagnetic energy which may be in the range of visible light, as one or more beams of such electromagnetic energy having a beam spot diameter resolution, at the surface of a material dispensed onto a carrier 124 on the moveable stage 104 on the order of 10 p diameter or less, and the inkjet type printer 112 is capable of dispensing droplets of bioink on the order of 50 to 500μ in diameter onto the carrier 124 surface, or a layer of material previously deposited thereon. Thus, by appropriate movement of the stage 104, and thus a carrier 124 positioned thereon, droplets of a desired biomaterial as a bioink in a liquid phase are dropped onto appropriate locations on the carrier, or onto locations on a previously formed layer of cured, uncured, or cured and uncured droplets, and as the stage 104 indexes in the X direction in the direction of the digital lithographic printer 114, to enable one or more beams of electromagnetic energy emitted therefrom to reach the dispensed liquid material layer and selectively cure it into a solid form. Here, as will be described further herein, the moveable stage is moveable in the X and Y directions of FIG. 1, and as each layer of slice of the material used to form the printed bio-material is dispensed and cured, the dispense station 108 and the cure station 100 are controllably moved away from the movable stage 104 by the thickness of the just cured slice by elevation thereof on the upright support 106.

Here, base 102 is a generally rectangular, in plain view, open frame 180 having opposed long walls 182, 184 facing each other across the width of the frame 180 and extending in the X direction of FIG. 1, opposed short walls 186, 188 facing each other across the length of the frame 180 and extending in the Y direction of FIG. 1, and connecting at their opposed ends, to opposed ends of the long walls 182, 184, and a plurality of stretchers 190 in the Y direction and extending between, and connected at their opposed ends, to the opposed long side walls 182, 184 at locations between the opposed short walls 186, 188. A guide rail 192 is provided to extend between and is connected at its opposed ends to the opposed short walls 186, 188, and it includes a bulbous, in cross section, upper a guide section 194. A support rail 196 likewise extends between and is connected at its opposed ends to the opposed short walls 186, 188, and at the opposed ends thereof are provided opposed first and second end support bosses 200, 202, through which extends a lead screw 204. A drive motor 206, for example a servo motor or stepper motor, is connected to an end of the lead screw 204 extending through the second end support boss 202, and is also connected to a motor support bracket 207 which is connected to the support rail 196 to hold the motor 206 stationary while an output shaft thereof, extending through the motor support bracket and connected to the one end of the lead screw 204, rotates. Lead screw 204 and guide rail 192 both extend in the X direction of FIG. 1, and parallel to one another. They together form a support for the moveable stage 104 to maintain the stage support surface 116 level, in other words in a plane which is perpendicular to the local direction of gravitation force.

Moveable stage 104 here is coupled to the upper guide section 194 of the guide rail 192 to be freely slidable, in the X direction of FIG. 1, thereon, and also receive the lead screw 204 therethrough. Here, moveable stage includes a first support portion 208, from the lower surface of which extends two support blocks 210, 212, each of which includes a lower surface having a reverse bulbous contour thereon which is contoured to receive the upper surface of the bulbous contour of the guide section 194 of guide rail 192 thereagainst, and at least one extending support 216 extending below the first support portion, and including a threaded lead screw opening 218 extending therethrough through which the lead screw extends, the threads on the outer surface of the lead screw 204 received and threaded into the threads in the threaded lead screw opening 218. As the lead screw 204 and guide rail 192 are spaced from one another in the Y direction, and extend in the X direction and parallel to one another in the light direction of each, the first support portion 208 of the moveable stage is supported at one side thereof in the Y direction by the support blocks 212, 214 slidably engaged against the guide section 194 of the guide rail 192, and at the other side thereof by the lead screw 204 extending through the threaded lead screw opening 218 in the extending support 216. By rotating the lead screw in a first direction (clockwise or counterclockwise when looking at the exposed end of the motor 206 from the X direction, the first support portion 208 moves away from the motor 206 because the rotation of the threads on the lead screw 204 cause the extending support to process therealong in that same direction. Reversing the rotation direction of the lead screw 204, the first support portion will move in the opposite X direction, i.e., toward the motor 206. By selecting the pitch of the mating threads on the exterior surface of the lead screw 204 and mating threads in the in the threaded lead screw opening 218 of the extending support, the movement in the X direction of the first support portion 208 in the X direction can be appropriately selected to meet the desired requirements for movement of the moveable stage 104 desirable for the printing application. For example, micrometer movements in the X direction per 360° rotation of the lead screw 204 are contemplated, and as the lead screw rotation can be controlled in the range of individual degrees on rotation thereof, even smaller controlled X direction movements of the moveable stage 104 are possible herewith.

As the moveable stage 104 is controllably moved in the X direction, to allow the entire upper surface thereof to be exposable to one of the bioink droplet dispensers of the dispense station 108, the moveable stage needs to also move in the Y direction of FIG. 1. Thus, the moveable stage 104 further includes at least one linear driven portion 232, for example a rack or a linear magnet structure, and at least one linear drive portion 230, for example a pinion gear drive to drive a pinion gear to move the rack with respect thereto, or a magnetic motor to move the linear magnet with respect thereto, to move the support portion 208 in the Y direction. Thus, by controlling the X and Y position of the support portion 208, and location on the stage support surface 116 can be positioned under any of one or more bioink dispensers in the dispense station 108.

To form a bioprinted member, a carrier 124, having a carrier base 118 and surrounding walls(s) 120 together surrounding a bioink accessible open top receiving volume 122 of the carrier 124, is provided to be located on the stage support surface 116 and positioned to receive liquid droplets of biomaterial from the dispense station 108 and move to position the resulting layer or slice of liquid material thereon into a position under the digital lithographic printer 114, without meaningful change in position of the liquid layer of biomaterial on the carrier 124, to receive electromagnetic radiation from the digital lithographic printer 114 to cure at least portions of the liquid material layer thereon into a solid form. Thus, the carrier 124 is mounted on the stage support surface 116 of the moveable stage 104, and the moveable stage 104 is scanned in the X and Y directions to position discrete areas of the interior surface of the carrier base 118 under an inkjet dispense 112 to receive droplets of the biomaterial thereon in discrete desired locations thereon, and then move the dispensed liquid, in substantially the same location on the carrier 124, or a layer of cured liquid already thereon, as it was dispensed to, to a location under the digital lithographic printer 114 to receive an appropriate dosage, as a function of exposure time and intensity, of electromagnetic energy capable of curing the liquid to a solid form.

Droplets of bioink to form a layer of bioink, and droplets of a sacrificial material, which can be cured into a solid form using the electromagnetic energy of the digital lithographic printer 114, are dispensed onto the carrier to form discrete layers or slices of the artificial organoid being built. Each layer has a specific thickness. To ensure process uniformity, the distance between the output optics of the digital lithographic printer 114 and the surface of the layer or slice on the carrier 124 to be cured, or the distance from the opening in a bioink dispenser and the surface of a layer or slice, needs to remain constant. Thus, the dispense station 108 and digital lithographic printer 114 are coupled to a support surface 116, and a riser lead screw 242 extending through a threaded opening (not shown) in the support plate 240 is rotationally supported on upright support 106, and a riser motor 244 is coupled thereto to cause rotation of the riser lead screw 242 to enable raising and lowering of the support plate 240, and thus the digital lithographic printer 114 and the surface of the layer or slice on the carrier 124 and the dispense station 108 with respect to the surface of a layer or slice of material on the carrier, or the carrier base 118 of the carrier 124 itself.

During the formation of the bioprinted matter herein, a three-dimensional matrix, composed of discrete layers or slices of different curable liquids which are dispensed from the inkjet dispense printer 112, is formed and selectively cured, wherein a plurality of sublayers of the final bioprinted matter, for example a mammalian kidney, are formed, one over the other, until the entire kidney is printed on the carrier 124. As the biomatter, here for example an artificial organoid such as an artificial kidney, is composed of a plurality of individual subelements, for example blood vessels, ureter tissue, and other biologically active elements, these vessels, tissues, and other structures will often extend through several of the sublayers or slices of initially, as dispensed, uncured, and then cured, liquid material dispensed onto, and cured sequentially on, the carrier 124. The organoid being formed, here a kidney, is formed pursuant to a design, in which the individual elements of the kidney, and their interaction with one another, are taken into consideration, and this design is converted into a printing file and a curing file, which represents a sequence of printable slices of the kidney having the thickness of a sub layer or slice thereof, the sublayer or slice having a thickness which is both formable using the inkjet type printer and which inherently possesses sufficient locational definition and stability of the bioink while moved on the carrier 124 from the location where the liquid is received to the location where the liquid is cured with the electromagnetic energy available from the digital lithographic device. Additionally, each printed and subsequently cured slice or sub-layer has to be properly aligned to the slices or sublayers thereadjacent, i.e., immediately above or immediately below, to enable features that extend between slides to be contiguously formed across or through the multiple slices or sublayers.

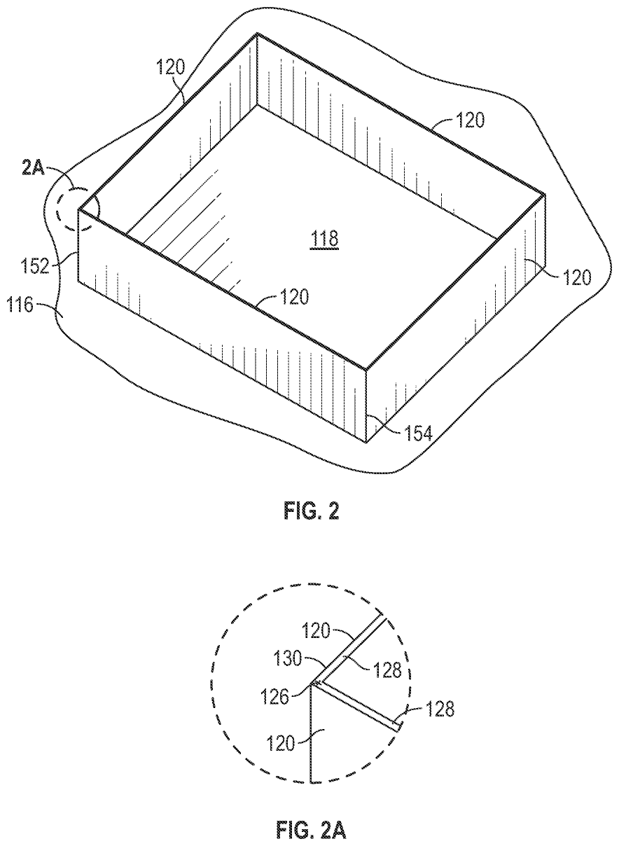

To aid in this alignment, carrier 124, here shown in FIG. 2, includes a rectangular carrier base 118 and four walls 120, each extending upwardly from a side of the carrier base 118, and at least one reference mark 126, here in the shape of an X, at the intersection location 130 of the upper walls 128 of adjacent walls 120 as shown in FIG. 2A, an enlarged view of the corner of the tray of FIG. 2. As will be described herein, the carrier 124 may include a plurality of such reference marks 126, for example at least two such marks at different intersections 130 of the upper walls 128 thereof, and the inkjet type printer 112 and the digital lithographic printer 114 each have a detection system including a visioning system, such as a camera, CCD, or other such imaging device, that is capable of locating the reference marks to determine the relevant location of the carrier, and thus locate, for purposes of dispensing droplets or curing a material thereon, any location on the upper surface of the carrier base 118 thereof, with respect to the operative elements of the inkjet type printer 112, the digital lithographic printer 114, and the moveable stage 104. Thus, the inkjet type printer 112 and digital lithographic printer 114 can direct the droplets of biomaterial, and the electromagnetic energy, respectively, to controllably selected locations of the carrier 124 and for the digital lithography printer, previously dispensed liquid, to sequentially create individual slices of the biomaterial with the internal components and features thereof in the proper alignment for the resulting designed biomatter.

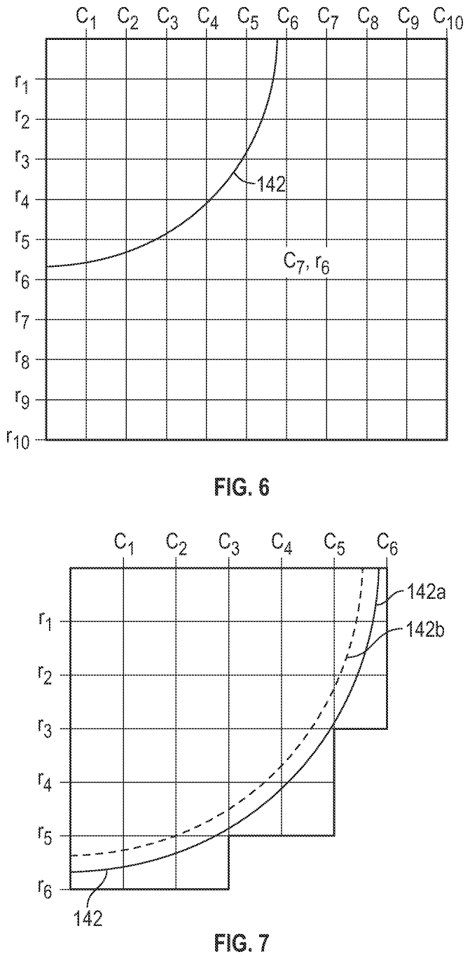

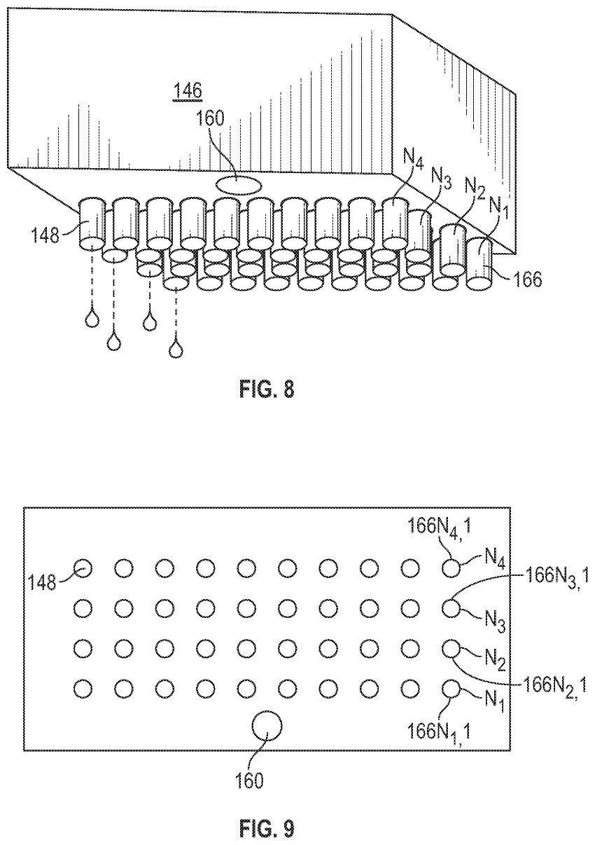

Referring now to FIGS. 8 to 10, a schematic views of an exemplary printhead of the inkjet printer 112, and the optical output array of the digital lithographic printer 114, are shown. To locate the ink of, i.e., print the organoid, as the carrier 124 is moving to the left in FIG. 1, optics in the inkjet type printer 112, here through a lens 160 on a printhead array 162 thereof, obtain an image of the reference marks on 126 on the carrier 124 on the ingress end thereof. A controller 164 is operatively connected to the inkjet head to selectively enable individual ones of a plurality of nozzles, here rows of Nozzles N1 to N4, where each nozzle 166 in a row N1 to N4 is coupled to the same source of liquid curable material, but the liquid curable material is different from nozzle row to nozzle row, to open to dispense a liquid bioink therefrom. Thus, for example, nozzle row N1 includes ten nozzles 166_{n1, 1} to 166_{n1, 10}, nozzle row N1 includes ten nozzles 166_{n1, 1} to 166_{n1, 10}, nozzle row N3 includes ten nozzles 166_{n3, 1} to 166_{n3, 10}, and nozzle row N4 includes ten nozzles 166_{4, 1} to 166_{n4, 10}. The pitch, or spacing, between adjacent ones of the nozzles 166 in any row N₁ to N₄ are approximately equal to, or smaller than, the area over which the liquid dispensed therefrom will spread out when it contacts an underlying surface, for example, the area of a grid locale such as locale c₇, r₆ of FIG. 6. For example here, assume that the nozzles of row N₁ dispenses a first bioink 140 containing lumen wall cells, the nozzles in row N₂ dispense a sacrificial material, and those in row and row N₃ dispense a third bioink 145 having a different cell than the lumen wall cell therein.

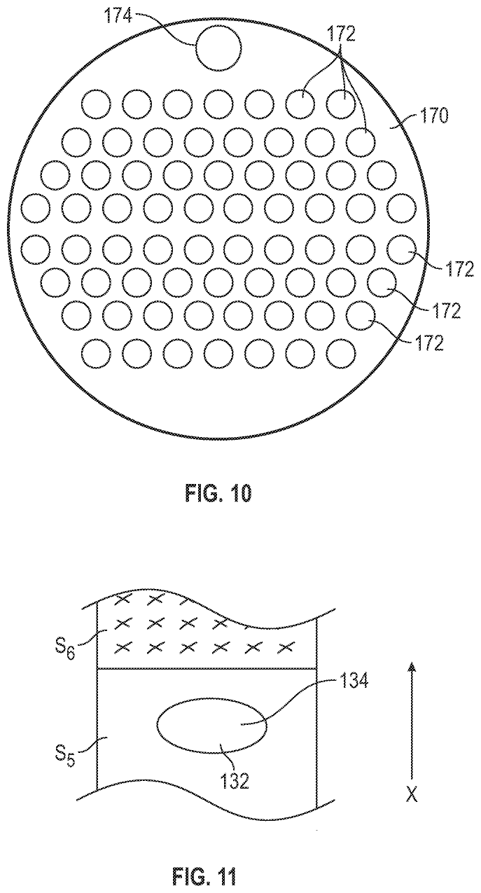

The digital lithographic printer 114 includes, as shown schematically in FIG. 10 which is a view of the stage facing side of a litho head 170 of the device, a plurality of electromagnetic energy beam outlets 172 and a beam synching optics pickup 174, connected to a camera (not shown) within the litho head 170. Controller 164 is likewise coupled to the camera and to the individual beam optics of the energy beam outlet 172, to selectively cause the energy beam outlets 172 to emit, or not emit, electromagnetic radiation, based upon which area of the slice of bioink passing thereunder intersects with the output of a particular energy beam. Thus, as each slice of liquid dispensed from the inkjet type printer 112 moves under the litho head 170, the individual electromagnetic energy beam outlets 172 selectively direct beam energy at specific areas of the slice. Here, the area of an electromagnetic energy beam emitted by any of the energy beam outlets 172 is less than the area that an individual droplet of bioink will cover on the base of the carrier 124 or a layer already formed thereon, for example, smaller than grid locale c7, r6 of FIG. 6. Thus, the resulting cross section of the bioink which is cured by the electromagnetic energy beam emitted by any of the energy beam outlets 172 is less than the area that an individual droplet of bioink has covered on the base of the carrier 124 or a layer already formed thereon, and thus a finer resolution, i.e., small sized feature, can be formed which is not limited in smallness of size by the size of the bioink droplet.

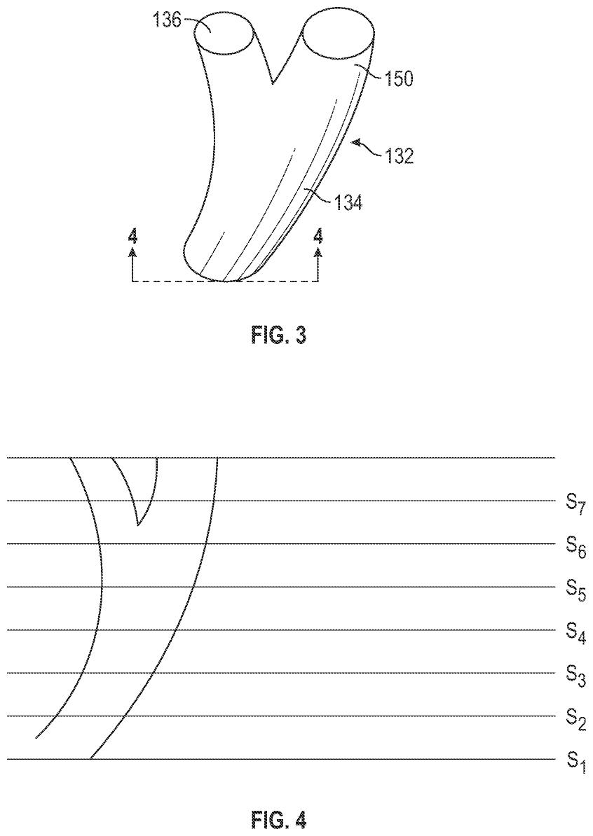

As a printing example, FIGS. 3 to 5 show an exemplary feature of a biomatter, here a branched vasculature or branched lumen 132 having a main lumen 134 portion, and a branch lumen 136 extending therefrom. In FIG. 3, the branched lumen is shown in an isometric view, whereas in FIG. 4 it is shown at section 4-4 of FIG. 3, for ease of demonstrating the slices or sublayers of bioink used to form the branched lumen 132. As shown in FIG. 4, the branched lumen 132 extends through multiple slices or sublayers of bio ink (and resultantly cured bioink), here slices S₁ to S₇ of a bioink to form the artificial organoid to be fabricated, each slice having a thickness which is both capable of being formed in liquid form on the carrier 124 or on a previously formed slice formed on the carrier 124, and being effectively cured using electromagnetic energy of the digital lithography printer 114. Here, only the branched lumen 132 of the artificial organoid being fabricated is shown for ease of illustration. However, it should be appreciated that each slice or sublayer of bioink, and thus of the resulting fabricated artificial organoid, is likely to include multiple portions of lumens and other organoid structural materials, which are aligned from slice to slice or sub-layer to sub-layer to fully fabricate the artificial organoid.

To print the branched lumen 132 of FIG. 3, for example the upper three slices thereof S₅ to S₇, different bioinks containing different biologically active or inactive materials must be employed. A first bioink 140 contains the bioactive material, i.e., bioactive lumen wall cells, from which the lumen wall is formed, here the lumen wall material. A second bioink 144 is also employed, which may also include biological cellular material used to form other features of the biomatter, filler material such as a sacrificial material, or combinations thereof. Once slice S₅ is formed in proper alignment with the underlying slice S₄, the stage support surface 116 is moved to the far right of the base 102 of FIG. 1, to a fixed potion, in the Y direction, which is a reference position for the stage support surface 116. Here, carrier 124 is mounted to the stage support surface 116. The carrier 124 here has opposed ends, for reference an ingress end 152 which is the end thereof which, when the stage support surface 116 initially moves to the left of FIG. 1, comes into place below the inkjet type printer 112, and an egress end 154, which is the last part of the carrier 124 to pass under the inkjet type apparatus 112 as the liquid forming a slice is deposited thereon or on a layer previously formed thereon.

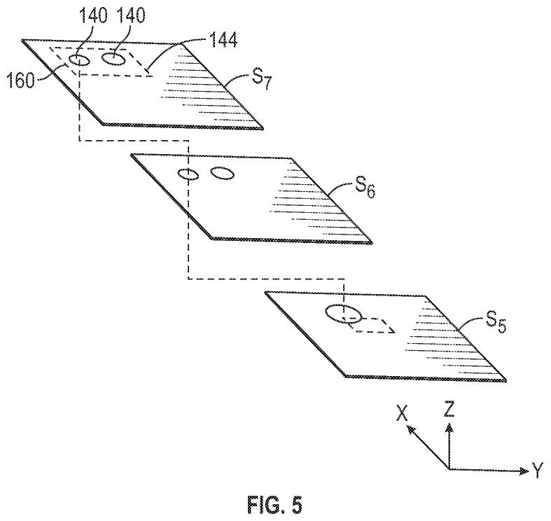

To print this branched lumen, and the material thereabout, a printing file is created based upon the design architecture of the artificial organoid, which creates individual ordered sub-layers or slices of the artificial organoid, and also identifies, in an X-Y plane of the slices or sublayers, for example the S5 to S7 of FIG. 5, individual printable locations on the slice as shown in FIG. 6. In FIG. 6, a plurality of rows r1-r10, and columns c1 to c10 are shown, and each represents the outline, and area, of the minimum resolution or size of a droplet of bioink that can be formed on the carrier 124 or a slice or sub-layer previously formed on the sublayer. The interstices of the columns and rows are shown as individual squares, each having sides of a length l. As droplets of bioink are dispensed onto the uppermost surface in the carrier 124, they will spread until contacting another drop, and their individual outlies will not necessarily be square. However, at the scale of the size of the droplets, a square is an acceptable mathematical compromise useful in the printing system hereof. For example, l may be on the order of 10 microns in length where the minimum droplet size will spread to a surface area of 10 microns across in the X-Y plane of the surface onto which it was dropped, such that each square, for example square r4, c6 is 100 microns in area in an X-Y plane.

For printing of the bioinks, here first and second bioinks 140, 144 to form the branched lumen 132 structure of FIGS. 3 to 5, each slice of the organoid is mathematically converted into a print file containing the bioink locales in column and row form for dispensing thereof into a grid pattern such as that shown in FIG. 6, except extending over the maximum X-Y dimensions of the widest portions of the artificial organoid being fabricated. Here, the grid of FIG. 6 is the small part of the slice S5 of FIG. 5 shown in dashed outline. Here, the outer surface 142 of the branched lumen at slice S5 is shown as an arc extending across the grid of FIG. 6, and in each square locale (row-column intersection) within the circumference of the outer wall, the first bioink is deposited by the inkjet printer 112, and in region outwardly thereof, the second bioink is deposited. Where the outer wall 142 passes through a grid locale box, the first bioink is deposited, as shown in FIG. 7, where only those grid locales having the first bioink therein are shown, the remaining grid locales of FIG. 6 containing the second bioink. Thus, for the branched lumen 132 of slice S5, the entire volume thereof circumscribed or surrounded by furthest extent or outer surface 142a of the contemplated outer wall 142 thereof will receive the bioink from which the outer wall is formed, although in the finished branched lumen the interior of the lumen inwardly of the outer wall 142 need remain open for the eventual flow of a liquid, blood, therethrough.

To locate the ink of, i.e., print the branched lumen 132 and surrounding material, as the carrier is moving to the left in FIG. 1, optics in the inkjet type printer 112 obtain an image of the carrier 124 and reference marks 126 on the egress end thereof through a lens 160 on a print head array 162 thereof. A controller 164 of the inkjet type printer is operatively connected to a plurality of nozzles, here rows of Nozzles N1 to N4, where each nozzle 166 in a row N1 to N4 is coupled to the same source of liquid curable material, but the liquid curable material is different from nozzle row to nozzle row. Thus, for example, nozzle row N1 includes ten nozzles 166_{n1, 1} to 166_{n1, 10}, nozzle row N1 includes ten nozzles 166_{n1, 1} to 166_{n1, 10}, nozzle row N3 includes ten nozzles 166_{n3, 1} to 166_{n3, 10}, and nozzle row N4 includes ten nozzles 166_{4, 1} to 166_{n4, 10}. the pitch, or spacing, between adjacent ones of the nozzles 166 in any row N₁ to N₄ are approximately equal to, or smaller than, the area over which the liquid dispensed therefrom will spread out when it contacts an underlying surface. For example here, assume that the nozzles or row N₁ dispenses first bioink containing lumen wall cells, row N₂ dispenses a second bioink 144 containing a different bioactive cell therein, and row N₃ dispenses the third material.

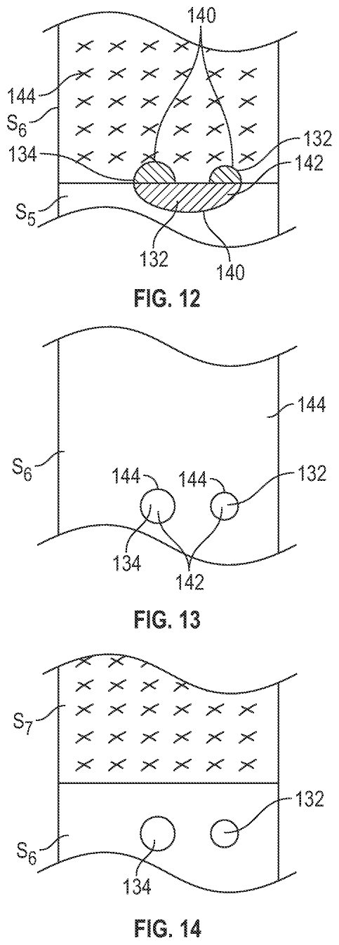

Referring to FIGS. 11 to 14, the printing of an exemplary slice S6 of FIG. 5 over exemplary slice S5 of FIG. 5 is shown, is shown, wherein the carrier 124 on which the slices S1 to S5 are already formed is moving in the X direction from the right to the left in Figure X such that the third material 144 has been dispensed in drops as a portion of slice S6 over the portion on slice S5 that does not include any portion of the branched lumen 132 therein. As the carrier 124 moves the slice S5 further toward the left hand side of Figure X, the location of the branch lumen 132 comes below the printhead array 162. Note, at slice S6 the branched lumen 132 branches such that a branch lumen 136 begins branching out from the main lumen 134 portion of branched lumen 132. Here, slice S5 includes cured first bioink 140 forming the lumen wall, surrounding a volume of uncured first bioink, and portions of cured and uncured second bioink in the region surrounding the cured first bioink 140. Thus, when the location of main lumen 134 is encountered, a portion of the liquid (uncured) first bioink 140 is printed over with the second bioink 144, and the lumen wall material of the first bioink 140 is dispensed in the lumen locations of the branch lumen 136 and main lumen 134, including area of the to be formed wall and the area circumscribed by the to be formed wall, each of which areas or regions are smaller than the main lumen 134 portion in slice S5. As the slice S5 continues to move, the material of the walls of the main lumen 134 and branch lumen 136 are surrounded in a field of second bioink 144.

After the bioink is dispensed, as the moveable stage moves the carrier in the X and Y direction to locate the appropriate nozzle to dispense the appropriate bioink over the grid locale directly therebeneath, portions of the just dispensed bioink will come below the energy beam outlets 172, which are selectively controlled to direct electromagnetic energy into all, or only a portion of, the bioink at any grid locale of the slice or sub-layer being printed and cured.

As the stage is passing under the inkjet type printer 112, it begins to pass under the digital lithographic printer 114, which includes, as shown in FIG. 10 which is a view of the stage facing side of a litho head 170 of the device, a plurality of electromagnetic energy beam outlets 172 and a beam synching optics pickup 174, connected to a camera (not shown) within the litho head. As each slice of liquid dispensed from the inkjet type printer 112 moves under the optics head, the individual electromagnetic energy beam outlets 172 direct beam energy at specific areas of the slice. For example, here the beam will define, within the layer of liquid lumen wall material of the first bioink 140, the actual lumen wall of the desired thickness (width in the X-Y plane of Figure X5), which may be less than the corresponding width of the lumen wall material of the first bioink 140 at that location. As shown in FIG. 7, where the portion of the portion of the slice of FIG. 7 which includes the first bioink 140 thereon, after curing of this material, the lumen outer wall 14a and lumen inner wall 14b define the radial limits of the cured portion of the first bioink 140, and that dimension is smaller than the side dimension of any grid locale that could be filled with a single droplet of bioink to the desired slice or sublayer thickness. Thus, although the minimum feature size that can be resolved using the inkjet type printer 112 is not the minimum feature size that can be resolved here, as the electromagnetic energy emanating from the beam outlets 172 can cure a region of the slice on the order of 10μ in diameter, significantly smaller than the possible minimum diameter of the liquid cell wall material 140 on the carrier 124 or a slice previously cured thereon. Thus, the electromagnetic beam selectively cures the liquid materials on the carrier or slice, and the portions of the liquid material which are not cured remain in place so to support the next layer of liquid to form the next slice, but like the sacrificial material, will be washed away from the final organ when the organ is fully printed with the liquid materials.

Once the entire layer S6 is printed and selectively cured, the moveable stage 104 returns to the far right of FIG. 1, and then starts again moving to the left in the X direction and positioning the slice S6 relative to the nozzles 166 to dispense the appropriate materials of the lumen wall material of the first bioink 140, sacrificial material and third material at their appropriate locations to form lumen walls of the main and branch lumens in the slice S7.

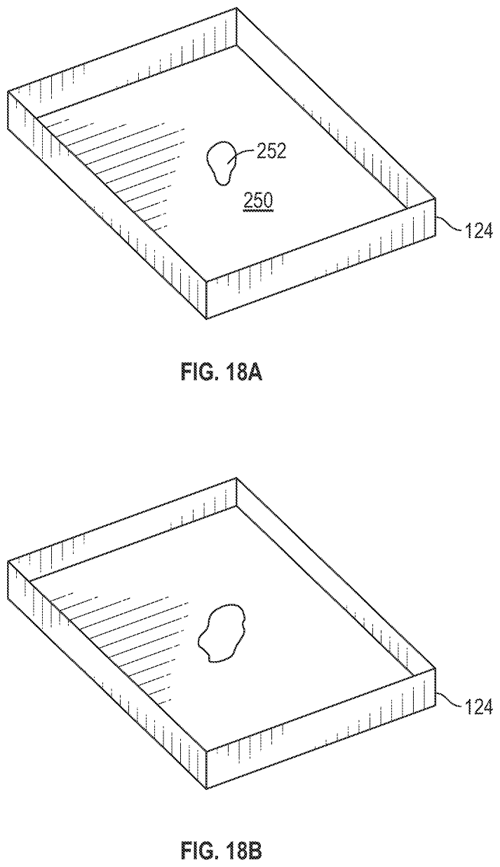

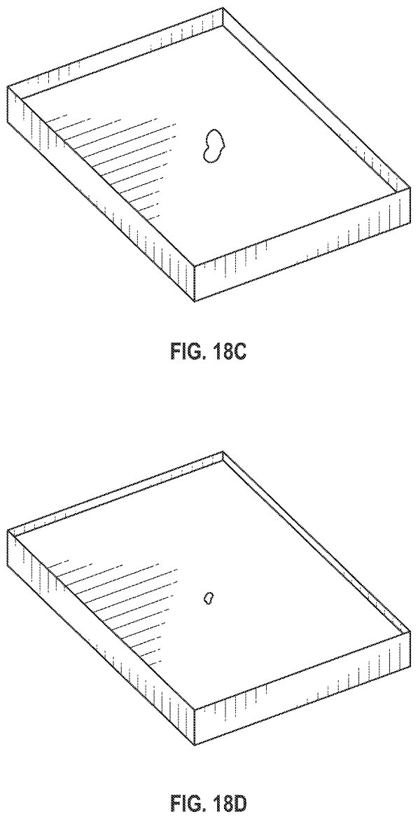

As the organoid is being fabricated on the carrier 124, the size of the organoid in each slice can, and likely will, change. Here, for example, the nozzles in the first three nozzle rows n1 to n3 are connected to sources of bioink containing living cells, and the nozzles in each row n1 to n3 are dedicated to a single type of bioink. Additionally, the nozzles of the fourth nozzle row are connected to a sacrificial material, i.e., a material which does not contain live cells and which is easily removed from the organoid being fabricated using a biocompatible washing material, such as water. Thus, as shown in FIGS. 18A to 18D, as the bioinks of nozzle rows n1 to n3, collectively bioinks 252, are deposited onto the carrier 124 or a sub layer or slice previously formed thereover, the sacrificial material 250 is dropped, as droplets, from the nozzles in nozzle row 4, such that the sacrificial material 250 forms a mechanical support layer to support the sides of the slice or sublayer of the bioinks 252, to help maintain the architectural integrity of the slices of the organoid as they are formed, to thereby help ensure that the use of the alignment system of the apparatus herein will properly align features of subsequent slices or sublayers with respect to the slices or sublayers therebelow.

Nozzle array 146 here is shown as including forty nozzles 148, laid out in four rows n1 to n4 of ten nozzles each. Each nozzle 148 in a row may, as previously described, be fluidly coupled to a common bioink or other type of curable liquid, or each nozzle 148 may be connected to a discrete type of curable liquid, which may be a bioink having living cells therein. Thus, where an organoid requires, for example, ten cell types to be fabricated, then for example ten sets of four nozzles 148 may configured so that each set dispenses a different curable liquid.

Controller 164 is provided, which may include a general purpose computer, capable of receiving an instruction set for execution of control of the motors 244, 206, digital lithographic printer 114, and the moveable stage Y direction motion, to selectively open any one or more nozzles simultaneously to dispense curable material therein to a known desired locale with respect to the reference marks 126 on the carrier, and likewise emit curing radiation to desired locations of the carrier relative to those reference marks 126, to control the dispensed locales of the curable materials, and the curing of that material, to form each slice of the organoid, and align each slice as it is being formed to the slice immediately below it.

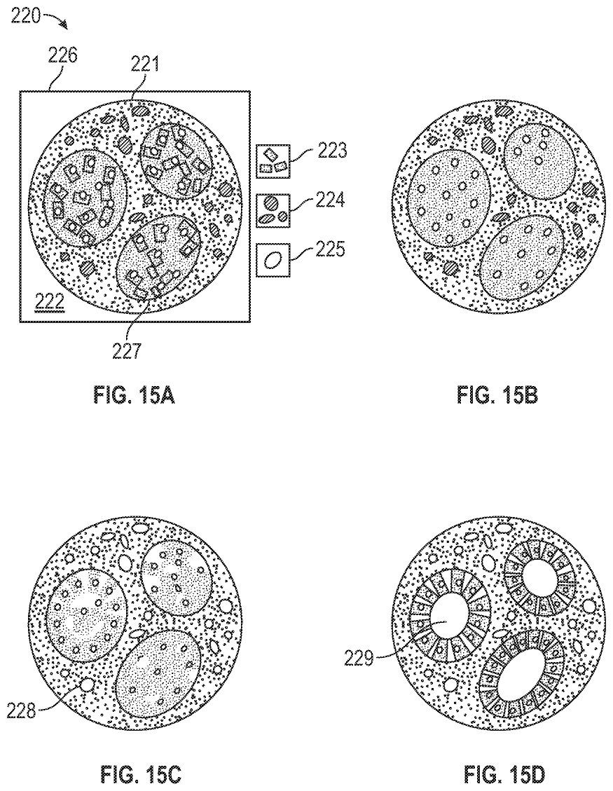

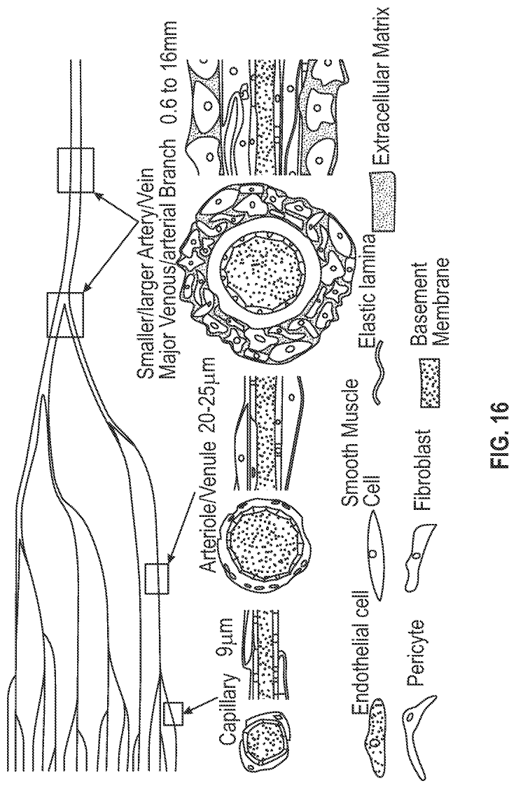

Referring now to FIGS. 15A-B, a cross sectional view of printed biomatter, for example of a 3D printed kidney is shown. In one embodiment, a portion of a luminal structured functional organoid 220, in this embodiment a kidney, is printed by the printing device dispense and cure station 100 on the carrier 222 onto which the biomatter is printed. Here, to print a 3D kidney, a structural design of a kidney is converted, using the controller hereof, into individual slices of kidney, including the location of different cell types thereof in each slice, such that individual slices of the kidney can be printed, one over the other, into a complete and functionable organ. Each slice therefore results in an organized biostructure, such as that shown in shown in FIG. 15D, which is an example of a printable portion of a biomaterial showing the exposed upper layer of the biomaterial which has been formed using 3D bio printing.

Here, three different kinds of bioinks including a biocompatible electromagnetic energy curable material and one of bioactive kidney cells 223, bioactive vasculature cells 224 and bioactive extracellular matrix cells 225 are provided and are dispensed and cured using the inkjet type printer 112 of FIG. 1 hereof to form a 3D printed kidney. These bioactive cells 223, 224 and 225 can form the functional cellular matrix of the resulting 3D printed kidney. To form this structure, the print head dispenses different bioinks each having one of the bioactive kidney cells 223, bioactive vasculature cells 224 and bioactive extracellular matrix cells 225 therein to print regions of bioink having kidney cell regions of ink, vasculature cell regions of ink, and extracellular matrix cell regions of ink in corresponding generally to the locations of kidney cells 223, vasculature cells 224 and extracellular matrix cells 225 in each slice of the kidney being printed. The droplet size 221 of the kidney, vasculature, or extracellular matrix cells 223, 224 and 225 which are dispensed are on the order of 10 um or greater. In other words, the smallest droplet size that can be dispensed onto the carrier 222 is 10 um, but larger droplet sizes can be dispensed on the order of 40-100 um, depending on the size of area of the slice required to be dispensed to that location or region of the slice being printed. An exterior sacrificial fluid 227 is also dispensed in droplets and is located around the exterior of the structure of each slice to serve as a support scaffold to support the outer structure of the kidney or other mammalian tissue as it is being printed.

Following droplet placement onto the carrier 222, or onto a previously printed and cured slice, the carrier 222 is moved under the curing station 110 and individual pulses of UV energy are directed, at preplanned intervals, into the just dispensed bioink in order to selectively cure the bioinks in the just dispensed slice. The droplets of bioink from which the kidney cells and extracellular matrix of the kidney are formed are generally of a size which is smaller than the area of the slice in which they are formed, and thus one, and often, multiple droplets of these bioink types are dispensed adjacent to one another to form the regions of the kidney cells and extracellular matrix in each slice. However, in some cases the actual final dimension where the bioactive cells in the bioinks is to remain is smaller than the smallest droplet size, and thus smaller than area of the slice where that bioink is dispensed. This is primarily an issue with the vasculature of the biomatter, here of the kidney being printed, wherein the vasculature wall is typically smaller in width, i.e., the wall thickness of the vessel or lumen from the inner surface to outer surface thereof, than the smallest area on which the bioink is present in the slice. However, here, the individual pulses of electromagnetic energy being emitted from the curing station 110 may be as small as submicron size in cross section, and thus an outer perimeter area of the vasculature cells to form a vasculature wall of bioactive vasculature cells 224 may, where required by the design of the biomatter, be cured with the electromagnetic energy of the curing station to have a cured area of a range below 10 um, for example, 1 um, and leaving liquid within, and in some cases exteriorly of, the resulting cured vasculature walls of each slice. A wavelength range of electromagnetic energy that will cure the polymer in the ink but not meaningfully deteriorate for cells is selected for this method. The cured polymer maintains the cells in place in the slice, allowing them to grow and replicate in place to form the biome and structure of the biomatter being printed, for example here, a mammalian kidney.

Following UV curing of the bioinks, areas of uncured bioink 228 remain in place to physically support the bioink of the next slice to be printed or dropped on the just cured slice. Additionally, around each of the slices, outside of the functional volume of the slice of kidney being dispensed, the sacrificial ink is dispensed, and then cured. This cured sacrificial ink is water soluble, and not rigid but help against movement by the walls of the carrier 222, such that the finished biomatter, here the kidney, becomes nearly encapsulated thereby, but is easily removed by peeling away the finished kidney from the sacrificial material 227. Additionally, the uncured bioink, and the cured sacrificial material potentially adhering to the outer surface of the finished kidney are 12 are flushed from the completed kidney at a wash station. As the sacrificial material 227 and uncured bioinks are water soluble, washing with water will allow the remaining liquid and cured sacrificial material to be removed without disrupting the cured structures of the biomatter. As the sacrificial fluid is washed out, the printed cured structures containing cells and other biocompatible materials necessary for cells to reorganize into biologically functional organ domains remain in situ. During the washing process gaps will be left in areas between cured portions of the biomatter, as well as shrinking of the sub domains of different cells types occurs and they can detach from surrounding areas, and liquid has been washed away to leave purposefully hollowed out regions or areas within the printed biomatter. The carrier 222 with the finished being printed and cured organ or biomatter is located in a conditioning device and incubated under appropriate conditions for cell growth so that internal gaps in the printed and cured biomatter are matured or repaired and sub domain structures therein are organized. As a result, material boundaries and structural boundaries are decoupled and are not limited to the size of the droplets.

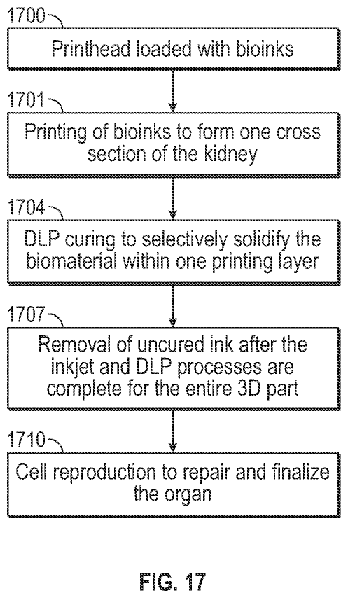

Referring now to FIG. 17, a flowchart useful for manufacture of a 3D bioprinted organoid is shown. At Act 1700 the bio-inks are provided to the print head array 162 of FIG. 1, and each nozzle, or each nozzle row N1 to N4 is couples to a different one of the bioinks, or other nozzle to bioink pairings are provided. Each nozzle 166 can have different physical (e.g., construction material, nozzle size, native resolution, viscosity/surface tension range) and process (temperature, meniscus pressure, frequency and waveform) parameters individually optimized for the jetting behavior (drop size, speed and shape, etc.) of each bioink. At Act 1701 the bio-inks are digitally deposited through single or multi pass inkjet printing to form a slice of the organoid. This step defines the gross material boundaries within the layer. Regions for very fine bio-structures (e.g., capillaries) generally require small drop size and high printing resolution, typically drop sizes resulting in areas of bioink of 40 um or less when deposited on the carrier 124 or a slice previously deposited thereon. Whereas areas for larger structures such as extracellular matrix (ECM) materials can be formed with more coarse resolution and larger drops to optimize process throughput.

At Act 1704, the fluidic slice of the organoid layer is selectively cured to form high resolution patterns, defining the structural boundaries therein. In this case, contours of the cured area should remain within their material boundary to ensure material homogeneity within the solidified, i.e., cured, structure, and a gap between adjacent two contours can range from a few micrometers to sub-millimeter sizes depending on inkjet resolution, material diffusion coefficients and material purity requirements. Here, for the vasculature of the organoid, a circular, elliptical, or other outline or shell is cured within the bioink containing the vasculature cells 224, while leaving the inner core thereof uncured. This will allow the cured shells to eventually grow into the blood vessels and capillaries of the kidney. At Act 1707 the uncured inks are removed through by transferring the substrate to a washing station to remove uncured formulations. This will result in gaps between different bio-components therein. At Act 1710 the substrate with the pre organ is placed into an incubator with appropriate conditions for growth, for example in 5% CO₂ at 37° C. The result of this incubation is a luminal structured functional organoid 220.

Referring now to FIG. 18, in FIG. 18a, as the initial slice of the organoid is formed, a region of bioinks 252 of a relatively small size is surrounded by a layer of sacrificial material 250 having the same thickness as the slice or sub-layer of the bioinks 252. As additional slices or sublayers of bioink 252 are formed, the slices or sub-layers have a larger cross section than the slices or sublayers below, and thus the surrounding layers of sacrificial material 250 supports the partial organoid from both the perimeter of the slice being printed, but from below as well, to maintain the architectural integrity of the organoid being fabricated. Then, as the fabrication of the organoid is substantially completed, the area of the slices or sublayers of the bioinks 252 become smaller as shown in FIG. 18C, but the entire organoid is structurally supported by the sacrificial material, to maintain the architectural integrity thereof. Eventually, as shown in FIG. 18D, the final slice or sublayer of bioink 252 of the organoid is printed, still surrounded by the sacrificial material. Thus, as a three dimensional organoid is fabricated, portions thereof which must be formed and which extend outwardly from the area of a previously printed slice or sub-layer may be supported, without impacting the functionality of the resulting organoid.

To facilitate understanding, identical reference numerals have been used, where possible, to designate identical elements that are common to the figures. It is contemplated that elements and features of one embodiment may be beneficially incorporated in other embodiments without further recitation.

What is claimed is:

1. A method of forming an artificial organoid having bioactive cells, comprising:
dispensing droplets of an electromagnetic energy curable bioink onto a surface to form a plurality of layers of the organoid in liquid form, wherein the droplets of bioink contain bioactive cell types therein of a structure to be formed in the organoid, and each droplet form a layer of bioink on the surface which is larger than a minimum feature size of a structure to be formed by curing the curable bioink; and
directing electromagnetic energy capable of curing the bioink having the bioactive cells into the layer of bioink, the electromagnetic energy having a beam width intersecting the bioink which is at least as small as the smallest feature of a structure to be formed in the organoid.

2. The method of claim 1, wherein the bioink is selectively cured using the electromagnetic energy.

3. The method of claim 2, further comprising forming multiple layers, one over the other, to form an organoid after completely forming all of the layers; and
selectively curing all of the layers.

4. The method of claim 2, further comprising forming multiple layers, one over the other, to form an organoids; and
incubating the organoid under appropriate cell growth conditions to mature or repair the organ.

5. The method of claim 1, wherein a bioink droplet forms a bioink layer on the surface as small as 10 um.

6. The method of claim 5, wherein the curing selectively solidifies the biomaterial to a size having a cured dimension less than 10 um in size.

7. The method of claim 1, wherein the surface is a previously formed, and cured layer of bioink.

8. An apparatus useful for forming an artificial organoid having bioactive cells, comprising:
a liquid dispensing device capable of dispensing droplets of an electromagnetic energy curable bioink onto a surface to form a slice of the organoid in liquid form, wherein the droplets of bioink contain bioactive cell types therein of a structure to be formed in the organoid, and a droplet forms an area larger than the smallest feature of the structure to be formed in the organoid; and
an electromagnetic beam directing device configured to direct electromagnetic energy capable of curing the bioink with the bioactive cells, the electromagnetic energy having a beam area intersecting the bioink which is smaller in width than the smallest feature of a structure to be formed in the organoid.

9. The apparatus of claim 8, further comprising a stage mounted on a moveable sub configured to selectively position the surface onto which the droplets are located below the liquid dispensing device.

10. The apparatus of claim 9, wherein the moveable sub is further configured to move the sate under the electromagnetic beam directing device.

11. The apparatus of claim 10, wherein the direction from the surface is positioned on, or in, a carrier, and the carrier is positioned on the stage.

12. The apparatus of claim 10, further comprising a controller operatively coupled to the moveable sub, the electromagnetic beam directing device and the liquid dispensing device.

13. The apparatus of claim 8, wherein the liquid dispensing device includes at least two nozzles selectively openable to release a curable liquid therefrom, wherein the plurality of nozzles include a first nozzle connected to a supply of a first liquid having a first cell type therein, and a second nozzle connected to a supply of a second liquid having a second cell type therein, and the first cell type and the second cell type are different.

14. The apparatus of claim 13, wherein, a droplet of the first liquid forms a layer on the surface which is smaller than a feature to be cured therein, and the second liquid forms a layer on the surface which is larger than a feature to be cured therein.

15. An apparatus for forming a three dimensional object by sequentially dispensing liquid, in droplet form, to form a first liquid layer on a surface, at least partially curing that first layer to form a first feature having a minimum dimension therein, sequentially dispensing liquid, in droplet form, to form a second layer on the first at least partially cured layer, and, at least partially curing that second layer to form a second feature having a minimum dimension therein, comprising:
a liquid dispensing device including at least one selectively openable nozzle connected to a supply of a liquid material, the nozzle capable of dispensing therefrom a droplet of at least a minimum volume, that droplet, when in contact with one of the surface of the at least partially cured first layer, having a thickness and an area over the one of the one of the surface of the at least partially cured first layer which is greater than the minimum dimension; and
an electromagnetic beam directing device outputting a beam of electromagnetic energy capable of curing, into a solid form, at least a portion of the material of the droplet, the beam of electromagnetic energy having a width dimension equal to, or less than, the minimum dimension.

16. The apparatus of claim 15, wherein the at least one selectively openable nozzle includes at least a first nozzle in fluid communication with a first liquid, and a second nozzle in fluid communication with a second nozzle, and at least one component of the first liquid is different from any component of the second liquid.

17. The apparatus of claim 16, wherein the first and second liquids each contain living cells, and the living cells in the first liquid are different than the living cells in the second liquid.

18. The apparatus of claim 17, wherein the at least one selectively openable nozzle includes a third nozzle connected to a supply of liquid having a component thereof different than the components of the first liquid and the second liquid.

19. The apparatus of claim 16, further comprising a moveable stage configured to move the surface first under the liquid dispensing device and then under the electromagnetic beam directing device.

20. The apparatus of claim 19, wherein the moveable stage is moveable in a first direction extending in the direction of from the liquid dispensing device to the electromagnetic beam directing device, and a second direction crossing the first direction.
